(19) United States
(12) Patent Application Publication
(10) Pub. No.: US 2003/0135163 A1
(43) Pub. Date: Jul. 17, 2003

(54) Veterinary syringe comprising a luer lock cannula attachment

(75) Inventor: Karl Schreijag, Rottweil (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(73) Assignee: Henke-Sass Wolf GmbH

(21) Appl. No.: 10/327,630

(22) Filed: Dec. 20, 2002

(30) Foreign Application Priority Data
Dec. 20, 2001 (DE) 201 20 662.5

(51) Int. Cl. A61M 5/00

(52) U.S. Cl. 604/240

(57) ABSTRACT

A veterinary syringe comprising a Luer lock cannula attachment provided at the injection-side end of a syringe barrel, wherein said attachment is provided as a hollow cylinder in its front region facing the cannula to be inserted, said hollow cylinder having its longitudinal axis aligned with the longitudinal axis of the syringe barrel of the veterinary syringe, and said attachment having its interior wall provided with a thread for reception of an attachable end of a cannula, which is provided with a suitable companion thread, and wherein a hollow cone, having an external diameter smaller than the diameter of the interior space of the hollow cylinder and comprising the injection channel for the medicament and protruding into the attachable end of the inserted cannula is arranged in the interior space of the hollow cylinder. The hollow cylinder being rotatable about its longitudinal axis relative to said hollow cone.

### BACKGROUND OF THE INVENTION

The invention relates to a veterinary syringe comprising a Luer lock cannula attachment provided at the injection-side end of a syringe barrel, wherein said attachment is provided as a hollow cylinder in its front region facing the cannula to be inserted, said hollow cylinder having its longitudinal axis aligned with the longitudinal axis of the syringe barrel of the veterinary syringe, and said attachment having its interior wall provided with a thread for reception of an attachable end of a cannula, which is provided with a suitable companion thread, and wherein a hollow cone comprising the injection channel for the medicament and protruding into the attachable end of the inserted cannula is arranged in the interior space of the hollow cylinder, said cone having an outside diameter which is smaller than the diameter of the interior space of the hollow cylinder.

These known Luer lock veterinary syringes are characterized by their relatively great robustness, in particular when performing serial vaccinations, and they withstand high stresses. This is achieved, in particular, by screwing the cannula onto the veterinary syringe using the known Luer lock connection and also by the perfect tightness provided by said connection. However, the desired stability of the Luer lock connection in the use of the veterinary syringe may be difficult to achieve when removing the cannula from the veterinary syringe, in particular after processing oily substances, because, in most cases, removal of said cannula from the syringe barrel will no longer be possible by hand, but will require the help of tools. This bears the risk, for example by slipping, of damaging the cannula, e.g. bending it, or of also injuring oneself.

Therefore, it is an object of the invention to achieve a most simple and risk-free use, in particular easy removal, of the cannula.

### SUMMARY OF THE INVENTION

For this purpose, according to the invention, the hollow cylinder of the above-described veterinary syringe is rotatable about its longitudinal axis relative to the hollow cone.

By this configuration of the attachment, allowing the hollow cylinder comprising the thread for screw insertion of the cannula attachment to be rotated relative to the hollow cone protruding into the cannula attachment, on the other hand, the cannula can be mounted on the veterinary syringe such that it is merely placed on the hollow cone, which advantageously protrudes a little further than the hollow cylinder, and is not screwed itself into the thread provided in the hollow cylinder of the attachment, but rather, the hollow cylinder of the attachment is rotated so that the cannula is securely and tightly placed on the hollow cone of the attachment.

Thus, this solution according to the invention actually offers multiple advantages, because, first, it is no longer required to rotate the cannula itself, so that the risk of damaging it or of injuring oneself is avoided. Second, the rotatable hollow cylinder provides better usability of the Luer lock connection as compared to the cannula, because the hollow cylinder has a larger diameter than the cannula or its attachment and, consequently, a greater torque may be applied during mounting, so that, in particular when dismounting the cannula, especially when the cannula is stuck, the connection between the cannula and the adapator may still be released by hand. Third, the position of the cannula, which is, in fact, determined by the oblique tip of its needle, may be freely selected relative to the veterinary syringe, because it may be fitted on in the desired position and then be mounted on the hollow cone without being rotated itself.

In an advantageous further embodiment of this solution, the hollow cone is secured on the end of the syringe barrel by means of a cylindrical base whose external wall is provided with a circumferentially extending groove into which a snap ring is inserted, the hollow cylinder being extended in a direction toward the syringe barrel and having a smooth-surface internal wall thereof supported on the external wall of the base of the hollow cone, said internal wall comprising a companion groove exactly opposite the groove in the external wall of the hollow cone, into which companion groove the snap ring also protrudes.

This particular embodiment of the attachment ensures that the rotatable hollow cylinder is undetachably connected with the hollow cylinder which is securely attached to the injectionside end of the syringe barrel or of the veterinary syringe.

In order to provide a particularly stable fit of the hollow cylinder on the hollow cone, the base of the hollow cone, on the one hand, and the hollow cylinder in its extended region, on the other hand, have larger outer or inner diameters than in the region provided for the attachment of the cannula.

The external wall of the hollow cylinder is conically shaped in its region designed for the attachment of the cannula, so as to provide the attachment with a pleasing shape as compared to the cannula to be placed thereon or to the attachable end of the cannula.

In order to facilitate handling of the hollow cylinder, in particular power transmission thereto, the hollow cylinder comprises an external surface which is provided with profiled gripping surfaces, which external surface may, in particular, also comprise specially designed gripping depressions.

### BRIEF DESCRIPTION OF THE DRAWINGS

The invention will be explained in more detail below, by way of example and with reference to the Figures, wherein:

FIG. 1 shows a cannula provided with a Luer lock attachment;

FIG. 2 shows the front part of a syringe barrel of a veterinary syringe provided with a Luer lock attachment; and

FIG. 3 shows the front part of the syringe barrel with a cannula placed thereon.

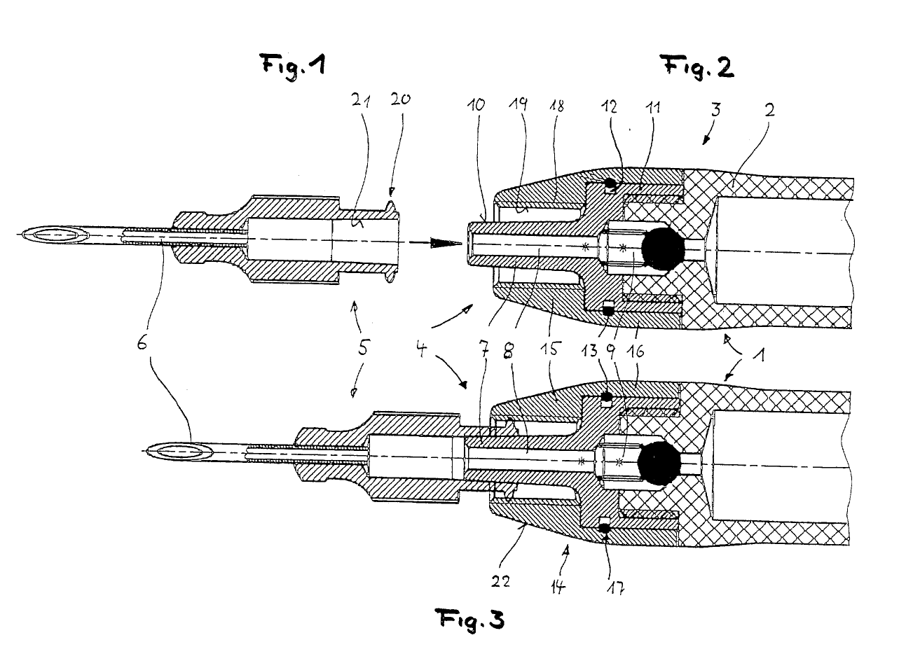

### DETAILED DESCRIPTION OF THE PREFERRED EMBODIMENT

The veterinary syringe 3, which is not shown completely in the Figures, but only with the injection-side end I of a syringe barrel 2, comprises an attachment 4 for the attachable end 5 of a cannula 6 to be placed therein.

The attachment 4 essentially has a two-part design and consists, first, of a hollow cone 7, which is securely attached to the syringe barrel 2 and comprises an injection channel 8 connected with the interior of the syringe barrel 2 via a valve device 9, which will not be described in any further detail. In this case, the hollow cone 7 comprises a fitting region 10, facing the attachable end 5 of the cannula 6 and of slightly conical design in its external wall region, and a base 11, which faces the syringe barrel 2, is provided for being mounted on said barrel, and has a larger diameter. Said base 11 is cylindrical, and its external wall is provided with a circumferentially extending groove 12 into which a snap ring 13 is inserted.

The thus-designed hollow cone 7 is surrounded by the second part of the attachment 4, namely the hollow cylinder 14, which comprises a front portion 15 and a rear portion 16 which differ in that the rear portion 16 has an internal diameter corresponding to the external diameter of the base 11 and is supported thereon in a rotatable, sliding manner. In this case, the internal wall of said rear portion 16 comprises a companion groove 17 directly opposite the groove 12 of the base 11, said companion groove 17 also having a snap ring 13 inserted therein. This ensures that the hollow cylinder 14, although rotatably supported on the base 11, cannot be pulled off said base.

In contrast thereto, the front portion 15 of the hollow cylinder 14 has a smaller internal diameter, which is, however, larger than the largest diameter of the conically designed fitting region 10 of the hollow cone 7. In particular, the cylindrical internal wall 18 comprises a thread 19 into which a companion thread 20 provided on the attachable end 5 of the cannula 6 may be inserted by screwing. Moreover, the attachable end 5 of the cannula 6 is provided with a conical internal wall region 21 adapted to the conically designed fitting region 10 of the hollow cone 7.

The two-part design of the attachment 4 allows the hollow cylinder 14 to be rotatably positioned on the hollow cone 7 or on its base 11. Due to the rotatability of the hollow cylinder 14, the cannula 6 now no longer needs to be screwed into the thread 19 of the hollow cylinder 14, but rather it suffices if the attachable end 5 of the cannula is placed on the conically designed fitting region 10 of the cone 7 and is made to abut against the thread 19 of the hollow cylinder 14 by means of the companion thread 20. Then, by rotating the hollow cylinder 14, the cannula 6 is drawn up on the hollow cone 7 until the conically designed fitting region 10 of the hollow cone 7 abuts closely against the internal wall region 21 of the attachable end 5 of the cannula 6, thus creating a tight connection between the cannula 6, on the one hand, and the attachment 4 of the veterinary syringe 3.

In order to facilitate actuation of the hollow cylinder 14, its external surface 22 is specially designed. Thus, it may comprise, for example, profiled gripping surfaces or may also be provided with gripping depressions.

The present invention may be embodied in other specific forms without departing from the spirit or essential attributes thereof, and it is, therefore, desired that the present embodiments be considered in all respects as illustrative and not restrictive.

I claim:

1. A veterinary syringe comprising a Luer lock cannula attachment provided at the injection-side end of a syringe barrel, wherein said attachment is provided as a hollow cylinder in its front region facing the cannula to be inserted, said hollow cylinder having its longitudinal axis aligned with the longitudinal axis of the syringe barrel of the veterinary syringe, and said attachment having its interior wall provided with a thread for reception of an attachable end of a cannula, which is provided with a suitable companion thread, and wherein a hollow cone, having an external diameter smaller than the diameter of the interior space of the hollow cylinder and comprising the injection channel for the medicament and protruding into the attachable end of the inserted cannula is arranged in the interior space of the hollow cylinder, characterized in that the hollow cylinder is rotatable about its longitudinal axis relative to said hollow cone.

2. The veterinary syringe as claimed in claim 1, characterized in that the hollow cylinder comprises an external surface provided with profiled gripping surfaces.

3. The veterinary syringe as claimed claim 1, characterized in that the hollow cylinder has an external surface which is provided with gripping depressions.

4. The veterinary syringe as claimed in claim 1, characterized in that the hollow cone is attached to the end of the syringe barrel by means of a base which is of cylindrical shape and whose external wall is provided with a groove extending over the periphery thereof, said groove having a snap ring inserted therein, and in that the hollow cylinder is extended in a direction toward the syringe barrel and contacts the external wall of the base of the hollow cone with a smooth internal wall, said internal wall having a companion groove which is arranged directly opposite the groove in the external wall of the hollow cone and into which the snap ring protrudes as well.

5. The veterinary syringe as claimed claim 4, characterized in that the hollow cylinder comprises an external surface provided with profiled gripping surfaces.

6. The veterinary syringe as claimed in claim 4, characterized in that the hollow cylinder has an external surface which is provided with gripping depressions.

7. The veterinary syringe as claimed in claim 4, characterized in that the base of the hollow cone, on the one hand, and the hollow cylinder in its extended region, on the other hand, have larger external and/or internal diameters than the region provided for attachment of the cannula.

8. The veterinary syringe as claimed in claim 7, characterized in that the hollow cylinder comprises an external surface provided with profiled gripping surfaces.

9. The veterinary syringe as claimed in claim 7, characterized in that the hollow cylinder has an external surface which is provided with gripping depressions.

10. The veterinary syringe as claimed in claim 7, characterized in that the external wall of the hollow cylinder is cone-shaped in the region provided for attachment of the cannula.

11. The veterinary syringe as claimed in claim 11, characterized in that the hollow cylinder comprises an external surface provided with profiled gripping surfaces

12. The veterinary syringe as claimed in claim 11, characterized in that the hollow cylinder has an external surface which is provided with gripping depressions.
